(19) United States
(12) Patent Application Publication
(10) Pub. No.: US 2021/0040536 A1
(43) Pub. Date: Feb. 11, 2021

(54) TARGET NUCLEIC ACID-DETECTING METHOD USING THREE-WAY JUNCTION STRUCTURE-INDUCED ISOTHERMAL AMPLIFICATION

(71) Applicants: KOREA ADVANCED INSTITUTE OF SCIENCE AND TECHNOLOGY, Daejeon (KR); BIONANO HEALTH GUARD RESEARCH CENTER, Daejeon (KR)

(72) Inventors: Hyun Gyu PARK, Daejeon (KR); Seoyoung LEE, Daejeon (KR); Hyo Yong KIM, Daejeon (KR); Hyowon JANG, Daejeon (KR)

(21) Appl. No.: 16/966,839

(22) PCT Filed: Jan. 30, 2019
(86) PCT No.: PCT/KR2019/001257

§ 371 Date: Jul. 31, 2020

(30) Foreign Application Priority Data
Jan. 31, 2018 (KR) 10-2018-0012156

(51) Int. Cl. C12Q 1/686

(52) U.S. Cl. CPC C12Q 1/686

(57) ABSTRACT

The present invention relates to an isothermal nucleic acid amplification technique based on a three-way junction structure that is formed as a ThIsAmp template; a ThIsAmp primer; and a nucleic acid are associated with each other through a hybridization reaction. Beyond the limitation of the conventional EXPAR technique, that is, the restricted application range that the EXPAR can be used only for detecting short target nucleic acids having a 3′-OH end, the method according to the present invention can detect target nucleic acids at excellent efficiency without being limited to kinds of target nucleic acids.

### TECHNICAL FIELD

The present invention relates to a method of detecting a target nucleic acid using ThIsAmp (Three-way junction structure-induced Isothermal Amplification), and more specifically to isothermal nucleic acid amplification based on a three-way junction structure in which a ThIsAmp template, a ThIsAmp primer and a target nucleic acid are bound to one another through a hybridization reaction.

### BACKGROUND ART

Nucleic acids function to store the genetic information of an organism and to control all metabolic activity of the organism through coding, decoding, regulation and expression of genetic material. In particular, nucleic acids have been used as important biomarkers for the diagnosis of diseases, since the presence or absence of a certain gene or mutation is directly or indirectly associated with the onset of various diseases such as diabetes, cancer and infectious diseases caused by pathogen infection. However, nucleic acids are present in a very small amount in vivo, so amplification of a target nucleic acid is inevitably required for disease diagnosis using the nucleic acid. In 1985, polymerase chain reaction (hereinafter, referred to as “PCR”) was first developed by Kary B. Mullis.

PCR is a nucleic acid amplification method that exponentially amplifies a certain target nucleic acid through a chain reaction of denaturation, annealing and extension of the target nucleic acid based on temperature control using a pair of primers, which are short DNA strands complementary to a target nucleic acid, and a heat-resistant DNA polymerase. However, PCR has a disadvantage in that it can be limitedly used only in places equipped with equipment such as large hospitals or specialized diagnostic centers, since an expensive temperature control device is required in order to realize temperature control for the PCR reaction. Recently, as the demand for the development of point-of-care testing (POCT) technology has increased, interest in alternative technologies capable of realizing miniaturization by overcoming the drawback of PCR, namely that a temperature control device is required, has increased.

In response to this technology trend, isothermal nucleic acid amplification methods capable of amplifying nucleic acids at a constant temperature without a temperature control process have been actively developed since the early 1990s, and examples thereof include nucleic-acid-sequence-based amplification (NASBA), helicase-dependent amplification (HDA), recombinase polymerase amplification (RPA), strand displacement amplification (SDA), loop-mediated isothermal amplification (LAMP), rolling circle amplification (RCA), and exponential amplification reaction (EXPAR). Among the various isothermal nucleic acid amplification methods, EXPAR has been regarded as a technique having high potential for use as a POCT technique since it has a target nucleic acid amplification efficiency of up to 10⁸ times within a short reaction time of about 30 minutes (Jeffrey Van Ness et al., Proc. Nat'l. Acad. Sci. USA, 100:4504, 2003). Specifically, EXPAR is a method of exponentially amplifying a double-stranded DNA product through the function of a restriction enzyme and a DNA polymerase after the hybridization reaction between the restriction enzyme and a template in which the restriction enzyme recognition base sequence (at the center of the template) and the base sequence complementary to the target nucleic acid (at both ends of the template) are modified. However, EXPAR has a disadvantage in that only a target nucleic acid having a short length and a 3′-OH end can be detected, since the template is amplified using the target nucleic acid as a primer. For this reason, EXPAR is mainly limitedly used for microRNA as the target material.

Accordingly, the present inventors endeavored to develop POCT technology that can be used without limitations on target nucleic acids. As a result, the present inventors found that, when an exponential amplification reaction of a double-stranded DNA product was implemented through the action of a cleavage enzyme and a DNA polymerase by introducing a system for producing a three-way junction structure induced by a double-probe-based target nucleic acid recognition reaction, excellent amplification efficiency can be obtained without limitations on a predetermined target nucleic acid length or a 3′ end functional group, which is a drawback of conventional EXPAR technology, and completed the present invention based on this finding.

### DISCLOSURE

### Technical Problem

It is an object of the present invention to provide a method for detecting a target nucleic acid using isothermal nucleic acid amplification without limitations on a predetermined target nucleic acid length or a 3′ end functional group.

### Technical Solution

In accordance with one aspect of the present invention, the above and other objects can be accomplished by the provision of a method for detecting a target nucleic acid, comprising (a) reacting a composition comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a target nucleic acid, (iv) a DNA polymerase, and (v) dNTP to produce double-stranded DNA, and (b) detecting the produced double-stranded DNA.

In accordance with another aspect of the present invention, provided is a method for detecting a target nucleic acid comprising (a) reacting a composition comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a target nucleic acid, (iv) a DNA polymerase, (v) dNTP and (vi) a cleavage enzyme to produce double-stranded DNA, and (b) detecting the produced double-stranded DNA.

In accordance with another aspect of the present invention, provided is a composition for isothermally amplifying a nucleic acid comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a target nucleic acid, (iv) a DNA polymerase, and (v) dNTP.

### DESCRIPTION OF DRAWINGS

FIG. 1 is a schematic diagram illustrating a reaction of a ThIsAmp method according to the present invention, more particularly, A showing that the ThIsAmp reaction does not proceed when a target nucleic acid is not present, and B showing, when a target nucleic acid is present, the production of a three-way junction structure and the subsequent recycling of the target nucleic acid and the ThIsAmp primer due to the action of a cleavage enzyme and a DNA polymerase (Pathway 1) and an exponentially amplified reaction of the final double-stranded DNA product (Pathway 2).

FIG. 2 shows the results of an experiment on the effectiveness of the ThIsAmp method of the present invention through a determination as to whether or not a fluorescence signal is generated under various reaction conditions (a: target nucleic acid; b: ThIsAmp template; c: ThIsAmp primer; d: ThIsAmp template+ThIsAmp primer; e: target nucleic acid+ThIsAmp primer; f: target nucleic acid+ThIsAmp template; g: target nucleic acid+ThIsAmp template+ThIsAmp primer) (M1: target nucleic acid (100 nM); M2: ThIsAmp template (100 nM); M3: ThIsAmp primer (100 nM)).

FIG. 3 shows the results of an experiment on the target nucleic acid detection sensitivity of the ThIsAmp method of the present invention (T_{threshold}: time to reach a threshold fluorescence signal value of a sample).

FIG. 4 shows the results of an experiment on single-base mismatch detection performance depending on the length of the base sequence complementary to target nucleic acid in the ThIsAmp template (T_{thre, 1bMM}: time to reach a threshold fluorescence signal value of a sample containing a single-base mismatched nucleic acid; T_{thre, PM}: time to reach a threshold fluorescence signal value of a sample containing a target nucleic acid).

FIG. 5 shows the results of an experiment on the target nucleic acid detection specificity of the present ThIsAmp method.

FIG. 6 is a schematic diagram of a ThIsAmp reaction using two pairs of ThIsAmp probes, A showing that the ThIsAmp reaction does not proceed when a target nucleic acid is not present, and B showing, when a target nucleic acid is present, the production of a three-way junction structure and the subsequent recycling of the target nucleic acid and the ThIsAmp primer due to the action of a cleavage enzyme and a DNA polymerase (Pathway 1) and an exponentially amplified reaction of the final double-stranded DNA product (Pathway 2).

### BEST MODE

Unless defined otherwise, all technical and scientific terms used herein have the same meanings as appreciated by those skilled in the field to which the present invention pertains. In general, the nomenclature used herein is well-known in the art and is ordinarily used.

The method for detecting a target nucleic acid using ThIsAmp according to the present invention is a diagnosis method capable of overcoming the disadvantages of conventional EXPAR. The ThIsAmp of the present invention is a beneficial method that is capable of detecting target nucleic acids without limitations on the length of the nucleic acid or the type of the functional group at the 3′ end, by overcoming the limited utilization range in that it can be used only for the detection of short target nucleic acids having a 3′-OH end, which is a limitation of conventional EXPAR technology. In addition, by improving the final double-stranded DNA amplification efficiency through recycling of target nucleic acid and ThIsAmp primer provided by the ThIsAmp technology according to the present invention, the ThIsAmp method of the present invention is capable of offsetting the effect of delaying the subsequent final double-stranded DNA product amplification that may occur due to the production of a three-way junction structure, which is the initial step of the present technology. As a result, the ThIsAmp method of the present invention realizes amplification efficiency comparable to those of the conventional EXPAR method and high specificity.

Accordingly, in one aspect, the present invention provides a method for detecting a target nucleic acid comprising (a) reacting a composition comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a target nucleic acid, (iv) a DNA polymerase, and (v) dNTP to produce double-stranded DNA, and (b) detecting the produced double-stranded DNA.

In the detection method of the present invention, the double-stranded DNA may be produced through the following procedure:

(a) binding (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, and (iii) a target nucleic acid through a hybridization reaction to form a three-way junction structure I; (b) extending the ThIsAmp primer in the three-way junction structure I using a DNA polymerase to form a three-way junction structure II, (c) producing a trigger from the three-way junction structure II using a cleavage enzyme and a DNA polymerase, (d) hybridizing the trigger produced in the step above with the trigger-complementary base sequence of the ThIsAmp template of the three-way junction structure II, and (e) producing a double-stranded DNA product by extending the trigger of (a) using the ThIsAmp template as a template by the DNA polymerase.

As used herein, the term “ThIsAmp template” is a template having a base sequence complementary to a target nucleic acid, a base sequence complementary to ThIsAmp primer, a cleavage enzyme recognition base sequence, and a trigger-complementary base sequence, which are sequentially connected to one another, and the term “ThIsAmp primer” is a primer having a base sequence complementary to a nucleic acid and a base sequence complementary to a ThIsAmp template.

As used herein, the term “trigger” refers to a short DNA strand (trigger) that is bound to and then separated from an upstream region of a ThIsAmp template as a template strand by a new DNA synthesis reaction.

In the present invention, the DNA polymerase may have strand displacement activity to displace DNA bound to a template.

In the present invention, the DNA polymerase can be used without limitation, as long as it is a DNA polymerase having strand displacement activity, and the DNA polymerase is preferably a Kienow Fragment, Vent (exo-) DNA polymerase, Est DNA polymerase, phi29 DNA polymerase or the like.

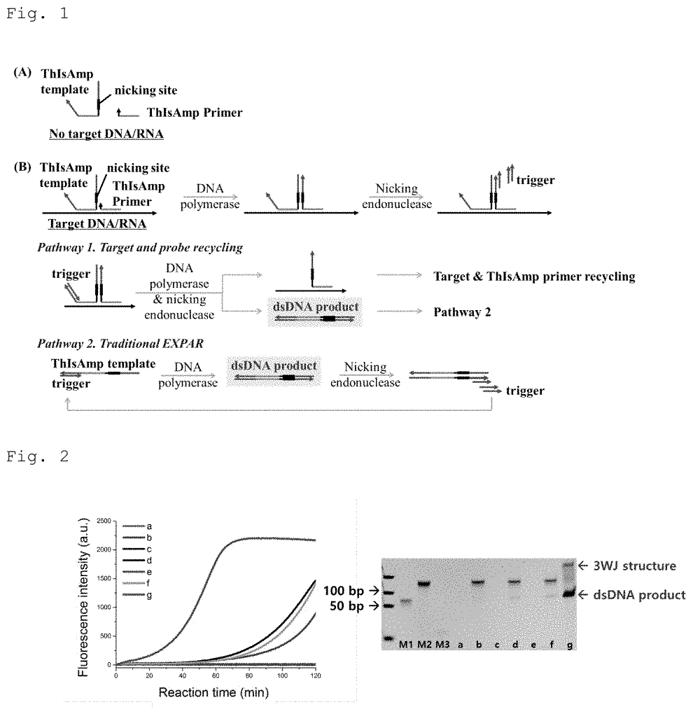

The method of the present invention is a method of detecting a target nucleic acid without limitations as to the length of the nucleic acid or the type of 3′ end functional group thereof by overcoming the limitations of conventional EXPAR technology through an isothermal nucleic acid amplification reaction based on a three-way junction structure. The method is capable of recognizing the target nucleic acid together with the ThIsAmp primer by introducing a ThIsAmp template that serves both as a three-way junction structure strand and as an EXPAR template. Also, the double-stranded DNA product can be exponentially amplified through the action of a cleavage enzyme and a DNA polymerase, thus the target nucleic acid can be detected without limitations as to the length of the nucleic acid or the type of 3′ end functional group (FIG. 1).

In the ThIsAmp method of the present invention, unlike an EXPAR method using a target nucleic acid as a primer, the trigger generated when the target nucleic acid is present acts as a primer for the EXPAR reaction, so the length of the target nucleic acid and the type of 3′ end functional group do not affect recognition of the target nucleic acid. After the three-way junction structure I is produced through the hybridization reaction of the target nucleic acid with the ThIsAmp template and the ThIsAmp primer, the ThIsAmp primer is extended due to the action of the DNA polymerase to form a three-way junction structure II having a cleavage enzyme recognition base sequence. When such a cleavage enzyme recognition base sequence is present, a single strand of double-stranded DNA is cleaved due to the activity of the cleavage enzyme present in the sample. Of the two cleaved strands produced in the above reaction, the strand forming a three-way junction structure with the target nucleic acid and the ThIsAmp template is used as a primer for new DNA synthesis to cause synthesis of new DNA due to the activity of the DNA polymerase present in the sample.

The DNA polymerase used in this reaction has the activity to displace the DNA strand bound to the upstream of the template strand (hereinafter referred to as “strand displacement activity”) during the DNA synthesis reaction. Accordingly, the new DNA synthesis reaction causes the short DNA strand (trigger) bound to the upstream of the ThIsAmp template as the template strand to be separated therefrom. Therefore, a large amount of a trigger is produced by conducting the cleavage and polymerization chain reaction of nucleic acids due to the combined action of the cleavage enzyme and DNA polymerase. The downstream of the ThIsAmp template also has a base sequence complementary to the produced trigger. For this reason, the ThIsAmp reaction proceeds through two different pathways.

First, the trigger binds to the downstream of the ThIsAmp template in the three-way junction structure, double-stranded DNA products are produced due to DNA synthesis and strand displacement activity of the DNA polymerase, and at the same time, the target nucleic acid and the extended ThIsAmp primer are separated therefrom. The separated target nucleic acid and the extended ThIsAmp primer react with a free ThIsAmp template to produce a new three-way junction structure II (Pathway 1). In addition, the trigger binds to the downstream of the free ThIsAmp template and then the double-stranded DNA product is exponentially amplified by cleavage and polymerization chain reaction using a cleavage enzyme and a DNA polymerase (Pathway 2). The above Pathway 1 and Pathway 2 can realize exponential amplification of a double-stranded DNA product.

Accordingly, in another aspect, the present invention provides a method for detecting a target nucleic acid comprising (a) reacting a composition comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a target nucleic acid, (iv) a DNA polymerase, (v) dNTP and (vi) a cleavage enzyme to produce double-stranded DNA, and

(b) detecting the produced double-stranded DNA.

In the detection method of the present invention, the double-stranded DNA may be produced in the following procedure:

(a) binding (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, and (iii) a target nucleic acid through a hybridization reaction to form a three-way junction structure I, (b) extending the ThIsAmp primer in the three-way junction structure I by a DNA polymerase to form a three-way junction structure II, (c) producing a trigger from the three-way junction structure II by a cleavage enzyme and a DNA polymerase, (d) hybridizing the trigger produced in the step above with the trigger-complementary sequence of the ThIsAmp template of the three-way junction structure II, and (e) producing a double-stranded DNA product by extending the trigger of (a) using the ThIsAmp template as a template by the DNA polymerase, (f) isolating the target nucleic acid and the extended ThIsAmp primer originally bound to the ThIsAmp template therefrom due to the reaction of (e), (g) reacting the isolated target nucleic acid and the extended ThIsAmp primer of step (f) with a free ThIsAmp template to form a triple junction structure II, and (h) conducting the hybridization reaction of the produced trigger with the free ThIsAmp template to produce double-stranded DNA through the activity of the DNA polymerase.

In the present invention, the method may further comprise producing a trigger in the double-stranded DNA generated in step (h) by the cleavage enzyme.

In the present invention, the DNA polymerase may have strand displacement activity to displace DNA bound to a template.

In another aspect, the present invention provides a composition for isothermally amplifying a nucleic acid comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a target nucleic acid, (iv) a DNA polymerase, and (v) dNTP.

In the present invention, the composition may further comprise a cleavage enzyme.

### EXAMPLES

Hereinafter, the present invention will be described in more detail with reference to the following examples. However, it will be obvious to those skilled in the art that the following examples are provided only for illustration of the present invention and should not be construed as limiting the scope of the present invention based on the subject matter of the present invention.

### Example 1: Establishment of Reaction Conditions of ThIsAmp

The process of preparing a ThIsAmp reaction solution of the present invention is as follows, but is not limited thereto. The reaction solution (final 20 μL) used in this example was prepared by adding 1.5 μL of dNTPs (10 mM each), 1 μL of a ThIsAmp template (1 μM), 1 μL of a ThIsAmp primer (100 nM), 1 μL of SYBR Green I (20X) and 2 μL of a target nucleic acid to a reaction buffer solution mixture (buffer A+buffer B). The reaction buffer A solution contained 20 mM Tris-HCl (pH 8.8), 10 mM KCl, 10 mM (NH4)₂SO₄, 2 mM MgSO₄, and 0.1% TritonX-100, and the reaction buffer B solution contained 25 mM Tris-HCl (pH 7.9), 50 mM NaCl, 5 mM MgCl₂, and 50 μg/mL of BSA. The reaction solution thus prepared was preheated at 55° C. for 10 minutes. Then, 1.2 μL of vent (exo-) DNA polymerase (0.5 unit/μL) and 0.4 μL of Nt.BstNBI (10 unit/μL) were added to the reaction solution and then the fluorescence signal generated from SYBR Green I was measured at 30-second intervals at 55° C. to analyze the amount of the double-stranded DNA that was ultimately produced.

The sequence of the ThIsAmp template, the ThIsAmp primer and the target nucleic acid used in this example are as follows.

ThIsAmp template (SEQ ID NO: 1):
TGC TCA AGG TGT GTC TAT G [symbol] T AAT GAC TCT CAT AAT
CAG CCG AAG CAG AGC GCA GGG TGC TCA AGG TGT GTC
TAT GT
ThIsAmp primer (SEQ ID NO: 2):
TGG ACG ACT TGA AAC AGC AGA GTT GAT TAT G
Target nucleic acid (SEQ ID NO: 3):
AGG TCT AGG GTG CGC TCT GCT TCG GCT CTC TGC TGT
TTC AAG TCG TCC AGC TCG TTC TT

(Boldface: trigger-complementary sequence; italics: Nt.BstNBI restriction enzyme recognition sequence; underlining: target nucleic acid-complementary sequence)

### Example 2. Verification of Effectiveness of ThIsAmp

A verification experiment was conducted by performing reactions under the following varied reaction conditions (a to g), using the same reaction conditions as in Example 1, except for the target nucleic acid, the ThIsAmp template, and the ThIsAmp primer.

a: Target nucleic acid;

b: ThIsAmp template;

c: ThIsAmp primer;

d: ThIsAmp template+ThIsAmp primer;

e: Target nucleic acid+ThIsAmp primer;

f: Target nucleic acid+ThIsAmp template;

g: Target nucleic acid+ThIsAmp template+ThIsAmp primer

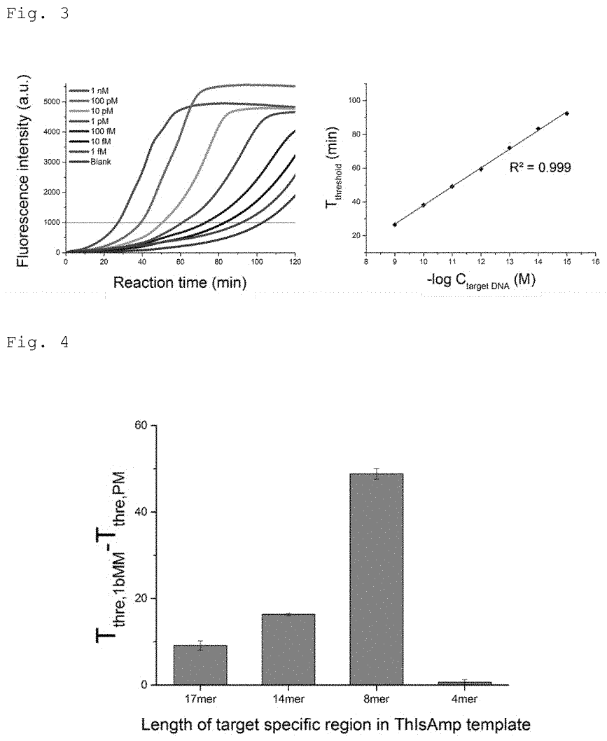

As shown in FIG. 2, the result showed that a remarkably strong fluorescence signal was generated under the reaction condition (g) including all of the target nucleic acid, the ThIsAmp template and the ThIsAmp primer. In addition, a verification test using electrophoresis showed that a three-way junction structure and a large amount of double-stranded DNA products were produced only under the reaction condition g.

### Example 3. Verification of Target Nucleic Acid Detection Sensitivity of ThIsAmp

An experiment for verifying the sensitivity of ThIsAmp was performed using the reaction conditions mentioned in Example 1.

Analytical samples containing target nucleic acids of various concentrations (1 fM to 1 nM) were prepared and then a ThIsAmp reaction was conducted. As shown in FIG. 3, the result showed that the limit of detection (LOD) of the target nucleic acid according to the present method was 78.1 aM. The results of this experiment demonstrated that the ThIsAmp method proposed in the present invention has performance comparable to that of the conventional EXPAR method.

### Example 4. Verification of Target Nucleic Acid Detection Specificity of ThIsAmp

A single-base mismatch detection performance experiment was performed depending on the target nucleic acid-complementary base sequence length (17 mer, 14 mer, 8 mer, 4 mer) in ThIsAmp template in order to verify the specificity of the ThIsAmp according to the present invention.

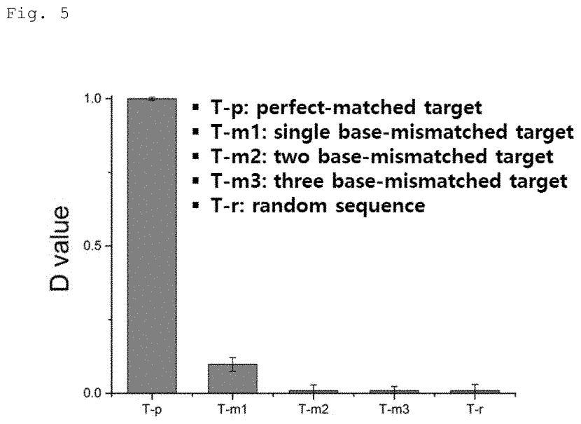

As shown in FIG. 4, the result showed that the single-base mismatch detection performance was the best when using a ThIsAmp template having a length of a base sequence complementary to a target nucleic acid of 8 mer.

The base sequence of each ThIsAmp template (17 mer, 14 mer, 8 mer, 4 mer) used in this example is as follows.

17mer (SEQ ID NO: 4):
TGC TCA AGG TGT GTC TAT G [symbol] T AAT GAC TCT CAT AAT
CAG CCG AAG CAG AGC GCA GGG TGC TCA AGG TGT GTC
TAT GT
14mer (SEQ ID NO: 5):
TGC TCA AGG TGT GTC TAT G [symbol] T AAT GAC TCT CAT AAT
CAG CCG AAG CAG AGC GGG TGC TCA AGG TGT GTC TAT
GT
8mer (SEQ ID NO: 6):
TGC TCA AGG TGT GTC TAT G [symbol] T AAT GAC TCT CAT AAT
CAG CCG AAG GGG TGC TCA AGG TGT GTC TAT GT
4mer (SEQ ID NO: 7):
TGC TCA AGG TGT GTC TAT G [symbol] T AAT GAC TCT CAT AAT
CAG CC GGG TGC TCA AGG TGT GTC TAT GT

(boldface: trigger-complementary sequence; italics: Nt.BstNBI restriction enzyme recognition sequence; and underlining: target nucleic acid-complementary sequence)

The target nucleic acid detection specificity experiment was conducted using the adopted ThIsAmp template. The result showed that the ThIsAmp method is capable of successfully detecting one to three base mismatches as well as a random nucleic acid sequence.

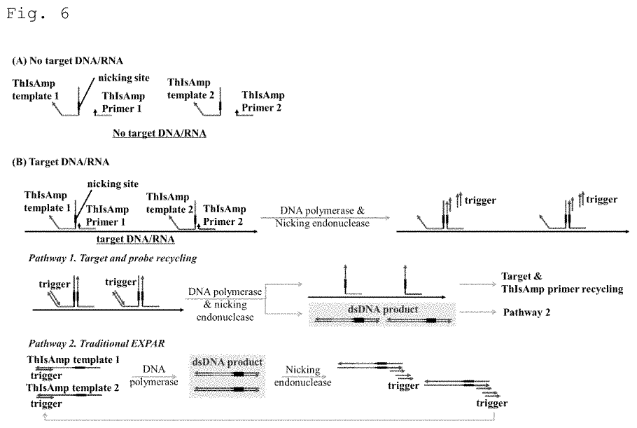

The results of the experiment on the target nucleic acid detection specificity are shown in FIG. 5, and the D value is a parameter indicating the ability to detect base mismatches from the target nucleic acid, and may be defined by the following Equation:

D value=(T_{thre,x}−T_{thre,0})/(T_{thre,P}−T_{thre,0})

(T_{thre,x}: time to reach a threshold fluorescence signal value of a sample containing various types of nucleic acids; T_{thre,0}: time to reach a threshold fluorescence signal value of a sample containing no target nucleic acid; T_{thre,P}: time to reach a threshold fluorescence signal value of a sample containing a target nucleic acid)

The results described above demonstrated that the ThIsAmp proposed in the present invention has excellent specificity.

### INDUSTRIAL AVAILABILITY

The present invention has an effect of enabling detection of target nucleic acids with high efficiency without limitation as to the type of target nucleic acids by solving the problem of limited utilization range of a conventional EXPAR technique, which is limited to the detection of target nucleic acids having a short length and a 3′-OH end.

Although specific configurations of the present invention have been described in detail, those skilled in the art will appreciate that the preferred embodiments are given for merely illustrative purposes in the description, and should not be construed as limiting the scope of the present invention. Therefore, the substantial scope of the present invention is defined by the accompanying claims and equivalents thereto.

### SEQUENCE LISTING FREE TEXT

An electronic file is attached.

1. A method for detecting a target nucleic acid comprising:
(a) reacting a composition comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a DNA polymerase, and (iv) dNTP with a sample containing a target nucleic acid to produce double-stranded DNA; and
(b) detecting the produced double-stranded DNA.

2. The method according to claim 1, wherein the DNA polymerase has strand displacement activity to displace DNA bound to a template.

3. A method for detecting a target nucleic acid comprising:
(a) reacting a composition comprising (i) a ThIsAmp template in which a base sequence complementary to a target nucleic acid, a base sequence complementary to a ThIsAmp primer, a cleavage enzyme recognition base sequence and a base sequence complementary to a trigger are sequentially linked to one another, (ii) a ThIsAmp primer having a base sequence complementary to the target nucleic acid and a base sequence complementary to the ThIsAmp template, (iii) a DNA polymerase, (iv) dNTP and (v) a cleavage enzyme with a sample containing a target nucleic acid to produce double-stranded DNA; and
(b) detecting the produced double-stranded DNA.

4. The method according to claim 3, wherein the DNA polymerase has strand displacement activity to displace DNA bound to a template.

5. A composition for isothermally amplifying a nucleic acid comprising:
(i) a ThIsAmp template in which a base sequence complementary to a target nucleic acid, a base sequence complementary to a ThIsAmp primer, a cleavage enzyme recognition base sequence, and a base sequence complementary to a trigger are sequentially linked to one another;
(ii) a ThIsAmp primer having a base sequence complementary to the target nucleic acid and a base sequence complementary to the ThIsAmp template;
(iii) a DNA polymerase; and
(iv) dNTP.

6. The composition according to claim 5, further comprising a cleavage enzyme.


SEQUENCE LISTING (7 sequences)

SEQ ID NO: 1 - DNA, 77 bp, Artificial Sequence
  Synthetic construct
tgctcaaggtgtgtctatgttaatgactctcataatcagccgaagcagagcgcagggtgctcaaggtgtgtctatgt

SEQ ID NO: 2 - DNA, 31 bp, Artificial Sequence
  Synthetic construct
tggacgacttgaaacagcagagttgattatg

SEQ ID NO: 3 - DNA, 59 bp, Artificial Sequence
  Synthetic construct
aggtctagggtgcgctctgcttcggctctctgctgtttcaagtcgtccagctcgttctt

SEQ ID NO: 4 - DNA, 77 bp, Artificial Sequence; identical to SEQ ID NO: 1
  Synthetic construct

SEQ ID NO: 5 - DNA, 74 bp, Artificial Sequence
  Synthetic construct
tgctcaaggtgtgtctatgttaatgactctcataatcagccgaagcagagcgggtgctcaaggtgtgtctatgt

SEQ ID NO: 6 - DNA, 68 bp, Artificial Sequence
  Synthetic construct
tgctcaaggtgtgtctatgttaatgactctcataatcagccgaaggggtgctcaaggtgtgtctatgt

SEQ ID NO: 7 - DNA, 64 bp, Artificial Sequence
  Synthetic construct
tgctcaaggtgtgtctatgttaatgactctcataatcagccgggtgctcaaggtgtgtctatgt